(19) United States
(12) Patent Application Publication
(10) Pub. No.: US 2005/0288257 A1
(43) Pub. Date: Dec. 29, 2005

(54) Novel stable compositions of water and oxygen sensitive compounds and their method of preparation

(76) Inventors: Douglass F. Taber, Newark, DE (US); Hui-Yin Li, Hockessin, DE (US)

Correspondence Address: Hui-Yin Li, One Innovation Way, Newark, DE, 19711, US

(21) Appl. No.: 11/166,937

(22) Filed: Jun. 23, 2005

Related U.S. Application Data
(60) Provisional application No. 60/583,054, filed on Jun. 25, 2004.

(51) Int. Cl. A61K 31/675

(52) U.S. Cl. 514/86

(57) ABSTRACT

The present application described a new formulation for oxygen and/or water sensitive compounds with an inert material such as paraffin. The new formulation provides stability for the oxygen and/or water sensitive compounds in the air and can be handled easily. The new formulation of the present invention is useful as ligands and/or catalysts for preparation of pharmaceuticals, agrochemical, other fine chemicals and other synthetic compounds.

This application is a continuation of U.S. provisional application 60/583,054 filed on Jun. 25, 2004.

### FIELD OF THE INVENTION

This invention relates to novel compositions of water and oxygen sensitive compounds and their method of preparation.

### BACKGROUND OF THE INVENTION

Many compounds, particularly substituted phosphine containing compounds and its related metal complexes are useful ligands and/or catalysts for production of pharmaceuticals, agrochemicals, and many other organic materials. These compounds can have decreased or lost the desired properties when handled in the air due to oxidation and/or hydrolysis in the presence of oxygen and moisture. Therefore, protection of these compounds from oxidation and/or hydrolysis is needed to ensure that the compounds can provide desired effects at the time of their ultimate use.

For example, 1,3-(bis(mesityl)-2-imidazolidinylidene)dichloro-(phenylmethylene)(tricyclohexylphosphine)ruthenium is widely known for its applications in organic and polymer synthesis. Despite the obvious utility of this catalyst, its routine use is hindered by special storage requirements. Prolonged exposure to air and moisture deactivates the complex. Therefore, it is typically stored and handled under inert atmosphere, such as in the nitrogen filled glove box.

In a similar instance, numerous chiral phosphine containing catalysts are useful homogeneous hydrogenation catalysts and have been used widely for preparation of pharmaceuticals, agrochemicals, flavors and other fine chemicals. Almost all these catalysts require protection from oxidation and/or hydrolysis to retain their desired activities.

Numerous other compounds, such as those compounds listed in Aldrich and/or Strem chemical catalogs are sensitive to oxidation and/or hydrolysis.

As the oxidative and hydrolytic damage of many compounds are obvious problems, the needs exist for effective and convenient means to prevent and/or slow the undesired oxidation and/or hydrolytic process. The needs also exist for convenient storage and handling of such compounds.

### SUMMARY OF THE INVENTION

This invention provides a stable composition for water and oxygen sensitive compounds comprising (a) at least one water and/or oxygen sensitive compound; and (b) at least one inert material with a melting point ranging from 20° C. to 250° C. The composition can contain from 0.1% to 99.9% of the water and/or oxygen sensitive compound and 99.9% to 0.1% of the inert material.

This invention also provides a method of preparing a composition of the present invention. This method comprises dissolving or suspending a water and/or oxygen sensitive compounds in a inert material with a melting point ranging from 20° C. to 250° C.

This invention also relates to the use a composition of the present invention in the preparation of pharmaceuticals, agrochemicals, flavors and other fine chemicals.

### DETAILED DESCRIPTION OF THE INVENTION

In the above recitations, the term “inert material” is a material, which is stable to its exposure to air and to the moisture in the air; and does not affect the desired activity of the water and/or oxygen sensitive compound. An embodiment of the inert material is paraffin, wax, fatty acids, or a polymer with a melting point ranging from 20° C. to 250° C.

Preferred inert material is paraffin.

Also preferred inert material is wax.

The term “water and oxygen sensitive compounds” includes, but not limited to, organic and inorganic compounds, and a complex of the organic compound with metals. The metal in the complex of the organic compound includes, but not limited to, Sc, Ti, V, Cr, Mn, Fe, Co, Ni, Cu, Zn, Y, Zr, Nb, Mo, Tc, Ru, Rh, Pd, Ag, Cd, In, Sn, Al, La, Hf, Ta, W, Re, Os, Ir, Pt, Au, Hg, and Eu.

An embodiment of the water and oxygen sensitive compound is an organophosphine compund of Formula I; 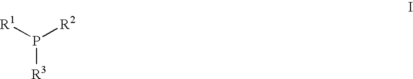
wherein
R¹, R² and R³ are each independently H; halogen; G; OG; NHG; NG₂; or C₁₋₃₂ alkyl, C₁₋₃₂ cycloalkyl; C₁₋₃₂ alkoxy, C₁₋₃₂ alkylamino, or C₁₋₃₂ dialkylamino each optionally substituted with one or more substituents selected from the group consisting of halogen, CN, NO₂, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₄-C₇ (alkyl)cycloalkylamino, C₂-C₆ alkoxycarbonyl, C₂-C₆ alkylaminocarbonyl, and C₃-C₈ dialkylaminocarbonyl; or
two of R¹, R² and R³ can be taken together to form a 5 to 8 membered ring optionally substituted with one or more substituents selected from the group consisting of halogen, CN, NO₂, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₄-C₇ (alkyl)cycloalkylamino, C₂-C₆ alkoxycarbonyl, C₂-C₆ alkylaminocarbonyl, and C₃-C₈ dialkylaminocarbonyl; said 5 to 8 membered ring contains from 0-2 heteroatoms selected from the group consisting of N, O, and S; and said 5 to 8 membered ring is optionally fused with 1 or 2 optionally substituted phenyl or naphthyl ring;
G is a phenyl ring, naphthyl ring, 5- or 6-membered heteroaromatic rings, aromatic 8-, 9- or 10-membered fused carbobicyclic ring systems, or aromatic 8-, 9- or 10-membered fused heterobicyclic ring systems, each ring or ring system optionally substituted with one or more substituents selected from the group consisting of C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₄-C₇ (alkyl)cycloalkylamino, C₂-C₆ alkoxycarbonyl, C₂-C₆ alkylaminocarbonyl, and C₃-C₈ dialkylaminocarbonyl;

A further embodiment of the water and oxygen sensitive compound is an organophosphine compound of Formula II; 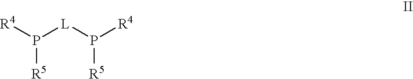
wherein
R⁴ and R⁵ are each independently phenyl, phenoxy, C₁₋₁₀ alkyl, C₁₋₁₀ cycloalkyl; C₁₀ alkoxy, C₁₋₁₀ alkylamino, or C₁₋₁₀ dialkylamino each optionally substituted with one or more substituents selected from the group consisting of halogen, CN, NO₂, C₁₋₈ alkyl, C₁₋₈ alkoxy, C₁₋₈ alkylamino, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₄-C₇ (alkyl)cycloalkylamino, C₂-C₆ alkoxycarbonyl, C₂-C₆ alkylaminocarbonyl, and C₃-C₈ dialkylaminocarbonyl; or
each R⁴ and R⁵ can be taken together to form a 4 to 8 membered ring optionally substituted with one or more substituents selected from the group consisting of halogen, OH, CN, NO₂, benzyl, C₁₋₈ alkyl, C₁₋₈ alkoxy, C₁₋₈ alkylamino, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₄-C₇ (alkyl)cycloalkylamino, C₂-C₆ alkoxycarbonyl, C₂-C₆ alkylaminocarbonyl, and C₃-C₈ dialkylaminocarbonyl; said 5-8 membered saturated, partially saturated or unsaturated ring contains from 0-2 heteroatoms selected from the group consisting of N, O, and S; and said 5-8 membered saturated, partially saturated or unsaturated ring is optionally fused with 1 or 2 optionally substituted phenyl or naphthyl ring; and
L is a 2 to 8 atom bridge containing C and/or O.

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers. “Alkoxy” includes, for example, methoxy, ethoxy, n-propyloxy, isopropyloxy and the different butoxy, pentoxy and hexyloxy isomers. “Alkylthio” includes branched or straight-chain alkylthio moieties such as methylthio, ethylthio, and the different propylthio and butylthio isomers. “Alkylsulfinyl” includes both enantiomers of an alkylsulfinyl group. Examples of “alkylsulfinyl” include CH₃S(O), CH₃CH₂S(O), CH₃CH₂CH₂S(O), (CH₃)₂CHS(O) and the different butylsulfinyl isomers. Examples of “alkylsulfonyl” include CH₃S(O)₂, CH₃CH₂S(O)₂, CH₃CH₂CH₂S(O)₂, (CH₃)₂CHS(O)₂ and the different butylsulfonyl isomers. “Cyanoalkyl” denotes an alkyl group substituted with one cyano group. Examples of “cyanoalkyl” include NCCH₂, NCCH₂CH₂ and CH₃CH(CN)CH₂. “Alkylamino”, “dialkylamino”, and the like, are defined analogously to the above examples. “Cycloalkyl” includes, for example, cyclopropyl, cyclobutyl, cyclopentyl, and cyclohexyl. The term “cycloalkylamino” includes the same groups linked through a nitrogen atom such as cyclopentylamino and cyclohexylamino.

The term “hetero” in connection with rings or ring systems refers to a ring or ring system in which at least one ring atom is not carbon and which can contain 1 to 4 heteroatoms independently selected from the group consisting of nitrogen, oxygen and sulfur, provided that each ring contains no more than 4 nitrogens, no more than 2 oxygens and no more than 2 sulfurs. The terms “heteroaromatic ring or ring system” and “aromatic fused heterobicyclic ring system” includes fully aromatic heterocycles and heterocycles in which at least one ring of a polycyclic ring system is aromatic (where aromatic indicates that the Hückel rule is satisfied). The heterocyclic ring or ring system can be attached through any available carbon or nitrogen by replacement of a hydrogen on said carbon or nitrogen.

The term “halogen”, either alone or in compound words such as “haloalkyl”, includes fluorine, chlorine, bromine or iodine. Further, when used in compound words such as “haloalkyl” or “halocycloalkyl”, said alkyl or cycloalkyl may be partially or fully substituted with halogen atoms which may be the same or different. Examples of “haloalkyl” include F₃C, ClCH₂, CF₃CH₂ and CF₃CCl₂. The terms “haloalkenyl”, “haloalkynyl”, “haloalkoxy”, “haloalkylthio”, and the like, are defined analogously to the term “haloalkyl”. Examples of “haloalkenyl” include (Cl)₂C═CHCH₂ and CF₃CH₂CH═CHCH₂. Examples of “haloalkynyl” include HC≡CCHCl, CF₃C≡C, CCl₃C≡C and FCH₂C≡CCH₂. Examples of “haloalkoxy” include CF₃O, CCl₃CH₂O, HCF₂CH₂CH₂O and CF₃CH₂O. Examples of “haloalkylthio” include CCl₃S, CF₃S, CCl₃CH₂S and ClCH₂CH₂CH₂S. Examples of “haloalkylsulfinyl” include CF₃S(O), CCl₃S(O), CF₃CH₂S(O) and CF₃CF₂S(O). Examples of “haloalkylsulfonyl” include CF₃S(O)₂, CCl₃S(O)₂, CF₃CH₂S(O)₂ and CF₃CF₂S(O)₂.

Examples of “alkylcarbonyl” include C(O)CH₃, C(O)CH₂CH₂CH₃ and C(O)CH(CH₃)₂. Examples of “alkoxycarbonyl” include CH₃C(═O), CH₃CH₂C(═O), CH₃CH₂CH₂C(═O), (CH₃)₂CHOC(═O) and the different butoxy- or pentoxycarbonyl isomers. Examples of “alkylaminocarbonyl” include CH₃NHC(═O), CH₃CH₂NHC(═O), CH₃CH₂CH₂NHC(═O), (CH₃)₂CHNHC(═O) and the different butylamino- or pentylaminocarbonyl isomers. Examples of “dialkylaminocarbonyl” include (CH₃)₂NC(═O), (CH₃CH₂)₂NC(═O), CH₃CH₂(CH₃)NC(═O), CH₃CH₂CH₂(CH₃)NC(═O) and (CH₃)₂CHN(CH3)C(═O).

The total number of carbon atoms in a substituent group is indicated by the “Cᵢ-Cⱼ” prefix where i and j are numbers from 1 to 32. For example, C₁-C₃ alkylsulfonyl designates methylsulfonyl through propylsulfonyl; C₂ alkoxyalkyl designates CH₃OCH₂; C₃ alkoxyalkyl designates, for example, CH₃CH(OCH₃), CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂.

The composition of the present invention can be prepared by adding the oxygen and/or water sensitive compounds to the molten inert material to give a homogeneous or heterogeneous mixture. For example, the oxygen and/or water sensitive compound can be added to the molten paraffin or wax to give a homogeneous or heterogeneous mixture. The homogeneous or heterogeneous mixture can be solidified upon cooling and can be cut into useful pieces and stored without precautionary protection from oxygen or water. The mixture can subsequently handled and weighted in the air.

The composition of the present invention can also be prepared by adding the molten inert material to the oxygen and/or water sensitive compound to give a homogeneous or heterogeneous mixture. For example, molten paraffin can be added to the oxygen and/or water sensitive compound to give a homogeneous or heterogeneous mixture. The homogeneous or heterogeneous mixture can be solidified upon cooling and can be cut into useful pieces for its use.

The composition of the present invention can also be prepared by heating a pre-mixture of the oxygen and/or water sensitive compound and the inert material. For example, paraffin and an oxygen and/or water sensitive compound can be mixed and subsequently heated to give a homogeneous or heterogeneous mixture. The homogeneous or heterogeneous mixture can be solidified upon cooling and can be cut into useful pieces for its use.

The composition of the present invention can also be prepared by mixing the oxygen and/or water sensitive compound and the inert material in a suitable organic solvent. For example, the paraffin and the oxygen and/or water sensitive compound solution in a suitable organic solvent can be mixed to give a homogeneous or heterogeneous solution. The homogeneous or heterogeneous solution can be solidified upon removing the organic solvent.

For example, the Grubbs catalyst, 1,3-(bis(mesityl)-2-imidazolidinylidene)-dichloro-(phenylmethylene)(tricyclohexylphosphine)ruthenium, can be weighed in a nitrogen atmosphere drybox and dispersed (11 wt %) in molten paraffin wax to give a homogeneous purple liquid. This mixture can be allowed to cool before removal from the drybox. The ruthenium complex-paraffin mixture can then be cut into useful pieces and stored without protection from laboratory air. This makes it convenient to weigh out small quantities of the otherwise easily dispersed ruthenium complex. The mixture can then be stored under normal laboratory conditions for many months without concern over the loss of catalytic activity. A sample prepared as described here can be stored for at least 22 months with no special storage precautions without loss of most of the catalyst's activity. For detailed reaction illustration of such prepared catalyst, see J. Org. Chem. 2003, 68, 6047-6048

### EXAMPLE

### Preparation of 1,3-(bis(mesityl)-2-imidazolidinylidene)-dichloro-(phenylmethylene)(tricyclohexylphosphine)ruthenium in paraffin

In a nitrogen atmosphere drybox, 1,3-(bis(mesityl)-2-imidazolidinylidene)-dichloro-(phenylmethylene)(tricyclohexylphosphine)ruthenium (Grubbs' catalyst 1) (1.27 g, 1.50 mmol) was combined in a 25 mL beaker with paraffin wax (paraffin wax for canning, mp=48-50° C., 8.73 g). The mixture was warmed to melting in a heating mantle and stirred with a glass rod to apparent homogeneity (one min). After cooling, the solid was removed from the drybox, broken into useful-sized pieces, and stored in a brown glass bottle without further protection.

In an embodiment of the present invention, the water and oxygen sensitive compounds include, but not limited to compounds in Table 1.

TABLE 1

| CAS Number | Chemical name |
|---|---|
| 169689-05-8 | N,N′-(1S,2S)-1,2-cyclohexanediylbis[2-(diphenylphosphino) benzamide; |
| 76740-45-9 | (1R,2R,3R,4S)-bicyclo[2.2.1]hept-5-ene-2,3-diylbis[diphenyl-, rel |
| | phosphine |
| 69227-47-0 | tris(3,5-dimethylphenyl) phosphine |
| 63589-61-7 | 1,2-ethanediylbis[(2-methoxyphenyl)phenyl phosphine |
| 55102-19-7 | chlorohydrotris(triphenylphosphine)-, (TB-5-13) ruthenium |
| 53204-14-1 | di-μ-chlorobis[1,2-ethanediylbis[diphenylphosphine-κP]]di rhodium |
| 15909-92-9 | 3,3′-(phenylphosphinidene)bis propanenitrile |
| 12150-46-8 | 1,1′-bis(diphenylphosphino) ferrocene |
| 7688-25-7 | 1,4-butanediylbis[diphenyl] phosphine |
| 6163-58-2 | tris(2-methylphenyl) phosphine |
| 5518-52-5 | tri-2-furanyl phosphine |
| 2622-14-2 | tricyclohexyl phosphine |
| 1314-80-3 | Phosphorus sulfide (P2S5) |
| 998-40-3 | tributyl phosphine |
| 443347-10-2 | 4,4′-bis[bis(3,5-dimethylphenyl)phosphino]-2,2′,6,6′-tetramethoxy-, (3S) |
| | 3,3′-bipyridine |
| 406680-94-2 | 1-(diphenylphosphino)-2-[(1R)-1- |
| | [(diphenylphosphino)methylamino]ethyl]-, (1R) ferrocene |
| 305818-67-1 | [(1,2,5,6-η)-1,5-cyclooctadiene][(2R,2′R,5R,5′R)-1,1′-(1,2- |
| | ethanediyl)bis[2,5-dimethylphospholane-κP]]-,, tetrafluoroborate(1−) |
| | rhodium(1+) |
| 290347-66-9 | 1,1′-bis[(2S,4S)-2,4-diethyl-1-phosphetanyl]-, ferrocene |
| 255884-98-1 | 1-[(R)-(dimethylamino)[2-(diphenylphosphino)phenyl]methyl]-2- |
| | (diphenylphosphino)-, (2R) ferrocene |
| 221012-82-4 | 4,4′-bis(diphenylphosphino)-2,2′,6,6′-tetramethoxy-, (3R) 3,3′-bipyridine |
| 213343-69-2 | Rhodium(1+), [(1,2,5,6-η)-1,5-cyclooctadiene][(2S,2′S,5S,5′S)-1,1′-(1,2- |
| | ethanediyl)bis[2,5-dimethylphospholane-κP]]-, salt with |
| | trifluoromethanesulfonic acid (1:1) |
| 213343-65-8 | [(1,2,5,6-η)-1,5-cyclooctadiene][(2S,2′S,5S,5′S)-1,1′-(1,2- |
| | ethanediyl)bis[2,5-dimethylphospholane-κP]] rhodium(1+), |
| | tetrafluoroborate(1−) |
| 213343-64-7 | [(1,2,5,6-η)-1,5-cyclooctadiene][(2S,2′S,5S,5′S)-1,1′-(1,2- |
| | phenylene)bis[2,5-diethylphospholane-κP]] rhodium(1+), |
| | tetrafluoroborate(1−) |
| 212210-87-2 | [(1R)-[1,1′-binaphthalene]-2,2′-diylbis[diphenylphosphine- |
| | κP]]dichloro[(1S,2S)-1,2-diphenyl-1,2-ethanediamine-κN,κN′]-, (OC-6- |
| | 13) ruthenium |
| 212143-24-3 | Ruthenium, [(1S)-[1,1′-binaphthalene]-2,2′-diylbis[diphenylphosphine- |
| | κP]][(2S)-1,1-bis(4-methoxyphenyl)-3-methyl-1,2-butanediamine- |
| | κN,κN′]dichloro-, (OC-6-14)- |
| 212143-23-2 | Ruthenium, [(1R)-[1,1′-binaphthalene]-2,2′-diylbis[diphenylphosphine- |
| | κP]]dichloro[(1R,2R)-1,2-diphenyl-1,2-ethanediamine-κN,κN′]-, (OC-6- |
| | 13)- |
| 210057-23-1 | [(1,2,5,6-η)-1,5-cyclooctadiene][(2R,2′R,5R,5′R)-1,1′-(1,2- |
| | phenylene)bis[2,5-dimethylphospholane-κP]] rhodium(1+), |
| | tetrafluoroborate(1−) |
| 208248-67-3 | N,N′-[(1R)-5,5′,6,6′,7,7′,8,8′-octahydro[1,1′-binaphthalene]-2,2′- |
| | diyl]bis[P,P-diphenyl] phosphinous amide |
| 205064-10-4 | [(1,2,5,6-η)-1,5-cyclooctadiene][(2S,2′S,5S,5′S)-1,1′-(1,2- |
| | phenylene)bis[2,5-dimethylphospholane-κP]] rhodium(1+), |
| | tetrafluoroborate(1−) |
| 203000-59-3 | [(2,3,5,6-η)-bicyclo[2.2.1]hepta-2,5-diene][(1R,1′R)-1,2- |
| | ethanediylbis[(1,1-dimethylethyl)methylphosphine-κP]] rhodium(1+), |
| | tetrafluoroborate(1−) |
| 194149-25-2 | P,P-bis(3,5-dimethylphenyl)-N,N-diethyl phosphinous amide |
| 192463-40-4 | tricyclo[8.2.2.24,7]hexadeca-4,6,10,12,13,15-hexaene-5,11- |
| | diylbis[diphenyl] phosphine, |
| 187682-63-9 | [(1,2,5,6-η)-1,5-cyclooctadiene][(2R,2′R,5R,5′R)-1,1′-(1,2- |
| | phenylene)bis[2,5-dimethylphospholane-κP]] rhodium(1+), salt with |
| | trifluoromethanesulfonic acid (1:1) |
| 185449-80-3 | N,N-dimethyl-, (11bS)-dinaphtho[2,1-d:1′,2′-f][1,3,2]dioxaphosphepin-4- |
| | amine, |
| 184095-69-0 | 1-[(1R)-1-[bis(3,5-dimethylphenyl)phosphino]ethyl]-2- |
| | (diphenylphosphino)-, (2R)- Ferrocene |
| 177750-25-3 | [(1,2,5,6-η)-1,5-cyclooctadiene][1,1′-(1,2-ethanediyl)bis[2,5- |
| | dimethylphospholane]-P,P′]-, [2R-[1(2′R*,5′R*),2α,5β]] Rhodium(1+), |
| | salt with trifluoromethanesulfonic acid (1:1) |
| 167416-28-6 | 1-(dicyclohexylphosphino)-2-[(1R)-1-(dicyclohexylphosphino)ethyl]-, |
| | (R)- Ferrocene |
| 166172-69-6 | bis[3,5-bis(trifluoromethyl)phenyl] phosphine |
| 158214-06-3 | α-D-Glucopyranoside, methyl, 2,6-dibenzoate 3,4-bis[bis(3,5- |
| | dimethylphenyl)phosphinite] |
| 155830-69-6 | 1-[(1R)-1-[bis(1,1-dimethylethyl)phosphino]ethyl]-2- |
| | (diphenylphosphino)-, (1R)- Ferrocene |
| 155806-35-2 | 1-[(1R)-1-(dicyclohexylphosphino)ethyl]-2-(diphenylphosphino)-, (R)- |
| | Ferrocene |
| 155613-52-8 | 4-chloro-, (11bR)-Dinaphtho[2,1-d:1′,2′-f][1,3,2]dioxaphosphepin, |
| 148461-14-7 | 2-[2-(diphenylphosphino)phenyl]-4,5-dihydro-4-(1-methylethyl)-, (4S)- |
| | Oxazole |
| 147253-67-6 | 1,1′-(1,2-phenylene)bis[2,5-dimethyl-, [2R-[1(2′R*,5′R*),2α,5β]] |
| | Phospholane |
| 145926-28-9 | [(1R)-[1,1′-binaphthalene]-2,2′-diylbis[diphenylphosphine- |
| | κP]]chloro[(1,2,3,4,5,6-η)-1-methyl-4-(1-methylethyl)benzene] |
| | ruthenium(1+), chloride |
| 145416-77-9 | [1,1′-binaphthalene]-2,2′-diylbis[bis(3,5-dimethylphenyl) phosphine |
| 142691-70-1 | 1,1′-bis(dichlorophosphino) ferrocene |
| 142184-30-3 | [(1,2,5,6-η)-1,5-cyclooctadiene][(2S,2′S,5S,5′S)-1,1′-(1,2- |
| | phenylene)bis[2,5-diethylphospholane-κP]] rhodium(1+), salt with |
| | trifluoromethanesulfonic acid (1:1) |
| 139139-93-8 | [(1S)-5,5′,6,6′,7,7′,8,8′-octahydro[1,1′-binaphthalene]-2,2′- |
| | diyl]bis[diphenyl] phosphine |
| 139139-92-7 | (1R)-[1,1′-binaphthalene]-2,2′-diylbis[dicyclohexyl]phosphine |
| 139139-86-9 | [(1R)-5,5′,6,6′,7,7′,8,8′-octahydro[1,1′-binaphthalene]-2,2′- |
| | diyl]bis[diphenyl]phosphine |
| 137219-86-4 | (1R)-[1,1′-binaphthalene]-2,2′-diylbis[bis(3,5-dimethylphenyl) phosphine |
| 137156-22-0 | Dinaphtho[2,1-d:1′,2′-f][1,3,2]dioxaphosphepin, 4-chloro-, (11bS)- |
| 136802-85-2 | bis(4-methoxy-3,5-dimethylphenyl) phosphinous chloride |
| 136779-28-7 | 1,1′-(1,2-phenylene)bis[2,5-diethyl-, (2S,2′S,5S,5′S)- Phospholane |
| 136779-27-6 | 1,1′-(1,2-ethanediyl)bis[2,5-diethyl-, (2S,2′S,5S,5′S)- Phospholane |
| 136779-26-5 | 1,1′-(1,2-ethanediyl)bis[2,5-dimethyl-, (2S,2′S,5S,5′S)- Phospholane |
| 136739-32-7 | [(1R)-[1,1′-binaphthalene]-2,2′-diylbis[diphenylphosphine-κP]]bis[(1,2,3- |
| | η)-2-methyl-2-propenyl] ruthenium |
| 136735-95-0 | 1,1′-(1,2-phenylene)bis[2,5-dimethyl-, (2S,2′S,5S,5′S)- Phospholane |
| 136705-77-6 | [(1,2,5,6-η)-1,5-cyclooctadiene][(2R,2′R,5R,5′R)-1,1′-(1,2- |
| | phenylene)bis[2,5-diethylphospholane-KP]] rhodium(1+), salt with |
| | trifluoromethanesulfonic acid (1:1) |
| 136705-75-4 | [(1,2,5,6-η)-1,5-cyclooctadiene][(2S,2′S,5S,5′S)-1,1′-(1,2- |
| | phenylene)bis[2,5-dimethylphospholane-κP]] rhodium(1+), salt with |
| | trifluoromethanesulfonic acid (1:1) |
| 136705-65-2 | 1,1′-(1,2-phenylene)bis[2,5-bis(1-methylethyl)-, (2R,2′R,5R,5′R)- |
| | Phospholane |
| 136705-64-1 | 1,1′-(1,2-phenylene)bis[2,5-diethyl-, (2R,2′R,5R,5′R)- Phospholane |
| 136705-62-9 | 1,1′-(1,2-ethanediyl)bis[2,5-diethyl-, (2R,2′R,5R,5′R)- Phospholane |
| 135139-00-3 | [(1S)-[1,1′-binaphthalene]-2,2′-diyl]bis[bis(3,5-dimethylphenyl)] |
| | phosphine |
| 134524-84-8 | Ruthenium, [[1,1′-binaphthalene]-2,2′-diylbis[diphenylphosphine- |
| | κP]]dichloro-, [SP-4-2-(S)]- |
| 133773-19-0 | Ruthenium, [[1,1′-binaphthalene]-2,2′-diylbis[diphenylphosphine]- |
| | P,P′]bis[(1,2,3-η)-2-methyl-2-propenyl]-, (S)- |
| 133545-17-2 | [(1S)-6,6′-dimethoxy[1,1′-biphenyl]-2,2′-diyl]bis[diphenyl] phosphine |
| 133545-16-1 | [(1R)-6,6′-dimethoxy[1,1′-biphenyl]-2,2′-diyl]bis[diphenyl] phosphine |
| 132071-87-5 | Ruthenium, [(1R)-[1,1′-binaphthalene]-2,2′-diylbis[diphenylphosphine- |
| | κP]]dichloro-, (SP-4-2)- |
| 130004-33-0 | [(1S)-[1,1′-binaphthalene]-2,2′-diylbis[diphenylphosphine- |
| | κP]]chloro[(1,2,3,4,5,6-η)-1-methyl-4-(1-methylethyl)benzene] |
| | ruthenium(1+), chloride |
| 129648-07-3 | 1,1′-(1,2-ethanediyl)bis[2,5-dimethyl-, (2R,2′R,5R,5′R)- Phospholane |
| 127797-02-8 | [[(4S,5S)-2,2-dimethyl-1,3-dioxolane-4,5-diyl]bis(methylene)]bis[bis(4- |
| | methoxy-3,5-dimethylphenyl) phosphine |
| 122977-44-0 | [[(4R,5R)-2,2-dimethyl-1,3-dioxolane-4,5-diyl]bis(methylene)]bis[bis(4- |
| | methoxy-3,5-dimethylphenyl) phosphine |
| 122708-97-8 | bis(4-methoxy-3,5-dimethylphenyl) phosphine |
| 121457-42-9 | (1S)-[1,1′-binaphthalene]-2,2′-diylbis[dicyclohexyl] phosphine |
| | ethanediyl]bis[diphenylphosphine-κP]]rhodium(1+), perchlorate |
| 64896-28-2 | [(1S,2S)-1,2-dimethyl-1,2-ethanediyl]bis[diphenyl] phosphine |
| 62793-31-1 | bis[(1,2,5,6-η)-1,5-cyclooctadiene] rhodium(1+), hexafluorophosphate(1−) |
| 62336-24-7 | (2-bromophenyl)diphenyl phosphine |
| 61478-29-3 | Pyrrolidine, 4-(diphenylphosphino)-2-[(diphenylphosphino)methyl]-, |
| | (2S,4S)- |
| 61478-28-2 | 1-Pyrrolidinecarboxylic acid, 4-(diphenylphosphino)-2- |
| | [(diphenylphosphino)methyl]-, 1,1-dimethylethyl ester, (2S,4S)- |
| 56977-92-5 | [(1,2,5,6-η)-1,5-cyclooctadiene][(1R, 1′R)-1,2-ethanediylbis[(2- |
| | methoxyphenyl)phenylphosphine-κP]] rhodium(1+), tetrafluoroborate(1−) |
| 56183-63-2 | tetrakis(1-methylethyl) phosphorodiamidous chloride |
| 55739-58-7 | 1,2-ethanediylbis[(2-methoxyphenyl)phenyl-, [R-(R*,R*)]- Phosphine |
| 55700-44-2 | 1-[(1R)-1-(dimethylamino)ethyl]-2-(diphenylphosphino)-, (2R)- |
| | Ferrocene |
| 55650-59-4 | 1-[(1S)-1-(dimethylamino)ethyl]-1′,2-bis(diphenylphosphino)-, (2S)- |
| | Ferrocene |
| 55650-58-3 | 1-[(1S)-1-(dimethylamino)ethyl]-2-(diphenylphosphino)-, (2R)- |
| | Ferrocene |
| 53111-20-9 | (2-methoxyphenyl)diphenyl] phosphine |
| 50763-75-2 | [[(4R,5R)-2,2-dimethyl-1,3-dioxolane-4,5- |
| | diyl]bis(methylene)]bis[diphenyl Phosphine, |
| 40244-90-4 | bis(1-methylethyl) phosphinous chloride |
| 37002-48-5 | [[(4S,5S)-2,2-dimethyl-1,3-dioxolane-4,5- |
| | diyl]bis(methylene)]bis[diphenyl] phosphine |
| 36042-94-1 | bis(2-methylphenyl) phosphinous chloride |
| 32993-05-8 | chloro(η5-2,4-cyclopentadien-1-yl)bis(triphenylphosphine) ruthenium |
| 32305-98-9 | [[(4R,5R)-2,2-dimethyl-1,3-dioxolane-4,5- |
| | diyl]bis(methylene)]bis[diphenyl] phosphine |
| 28240-69-9 | 1,2-ethanediylbis phosphonous dichloride |
| 25979-07-1 | (1,1-dimethylethyl) phosphonous dichloride |
| 25235-15-8 | (1-methylethyl) phosphonous dichloride |
| 18437-78-0 | tris(4-fluorophenyl) phosphine |
| 16941-11-0 | hexafluoro phosphate(1−), ammonium |
| 16523-54-9 | dicyclohexyl phosphinous chloride |
| 15873-72-0 | dicyclohexyl phosphinic chloride |
| 15529-49-4 | dichlorotris(triphenylphosphine) ruthenium |
| 15475-27-1 | diphenyl phosphine, potassium salt |
| 14973-89-8 | Rhodium, bromotris(triphenylphosphine)-, (SP-4-2)- |
| 14694-95-2 | Rhodium, chlorotris(triphenylphosphine)-, (SP-4-2)- |
| 14647-23-5 | Nickel, dichloro[1,2-ethanediylbis[diphenylphosphine-κP]]-, (SP-4-2)- |
| 14221-02-4 | Platinum, tetrakis(triphenylphosphine)-, (T-4)- |
| 14221-01-3 | Palladium, tetrakis(triphenylphosphine)-, (T-4)- |
| 13965-03-2 | dichlorobis(triphenylphosphine) palladium |
| 13685-23-9 | bis(3-methylphenyl) phosphinous chloride |
| 10150-27-3 | 2-[2-(diphenylphosphino)ethyl] pyridine |
| 10025-87-3 | Phosphoric trichloride |
| 7719-12-2 | Phosphorous trichioride |
| 6737-42-4 | 1,3-propanediylbis[diphenyl] phosphine |
| 5518-62-7 | 1,2-ethanediylbis phosphine |
| 4848-43-5 | 2-(diphenylphosphino) ethanamine |
| 4541-02-0 | diphenyl phosphine, lithium salt |
| 3487-44-3 | methylenetripheny phosphorane |
| 3348-44-5 | tetramethyl phosphorodiamidous chloride |
| 2844-89-5 | cyclohexyl phosphonous dichloride |
| 1663-45-2 | 1,2-ethanediylbis[diphenyl] phosphine |
| 1636-14-2 | N,N,N′,N′-tetraethyl-P-phenyl phosphonous diamide |
| 1608-26-0 | hexamethyl phosphorous triamide |
| 1498-40-4 | ethyl phosphonous dichloride |
| 1079-66-9 | diphenyl phosphinous chloride |
| 1069-08-5 | diethyl phosphoramidous dichloride |
| 1038-95-5 | tris(4-methylphenyl) phosphine |
| 855-38-9 | tris(4-methoxyphenyl) phosphine |
| 829-85-6 | diphenyl phosphine |
| 829-84-5 | dicyclohexyl phosphine |
| 819-19-2 | bis(1,1-dimethylethyl) phosphine |
| 811-62-1 | dimethyl phosphinous chloride |
| 686-69-1 | diethyl phosphinous chloride |
| 685-83-6 | tetraethyl phosphorodiamidous chloride |
| 676-83-5 | methyl phosphonous dichloride |
| 644-97-3 | phenyl phosphonous dichloride |
| 638-21-1 | phenyl phosphine |
| 603-35-0 | triphenyl phosphine |
| 589-57-1 | Phosphorochloridous acid, diethyl ester |
| 121-45-9 | Phosphorous acid, trimethyl ester |
| 1305-99-3 | Calcium phosphide; |
| 12261-30-2 | Cyclopentadienyl(triethylphosphine)copper (I); |
| 135707-05-0 | Trimethylphosphine(hexafluoroacetylacetonato)copper (I); |
| 12063-98-8 | Gallium phosphide (99.999%-Ga) PURATREM; |
| 15529-90-5 | Chlorotriethylphosphine gold (I); |
| 15278-97-4 | Chlorotrimethylphosphine gold (I); |
| 14243-64-2 | Chlorotriphenylphosphine gold (I); |
| 22398-80-7 | Indium phosphide (99.999%-In) PURATREM; |
| 14871-41-1 | Chlorocarbonylbis(triphenylphosphine)iridium (I); VASKA'S |
| | COMPLEX |
| 583844-38-6 | ((4S,5S)-(−)-O-[1-Benzyl-1-(5-methyl-2-phenyl-4,5-dihydro-oxazol-4-yl)- |
| | 2-phenylethyl]-dicyclohexylphosphinite)(1,5-COD)iridium (I) tetrakis- |
| | (3,5-bis(trifluoromethyl)phenyl)borate; |
| 17250-25-8 | Hydridocarbonyltris(triphenylphosphine)iridium (I); |
| 405235-55-4 | ((4S,5S)-(+)-O-[1-Benzyl-1-(5-methyl-2-phenyl-4,5-dihydro-oxazol-4- |
| | yl)-2-phenylethyl]-diphenylphosphinite)(1,5-COD)iridium (I) tetrakis- |
| | (3,5-bis(trifluoromethyl)phenylborate; |
| 64536-78-3 | (Tricyclohexylphosphine)(1,5-cyclooctadiene)(pyridine)iridium |
| | (I)hexafluorophosphate; CRABTREE'S CATALYST |
| 84680-95-5 | 1,1′-Bis(di-t-butylphosphino)ferrocene; |
| 494227-35-9 | (S,S)-(+)-2,2′-Bis[(R)-(N,N-dimethylamino)(phenyl)methyl]-1,1′- |
| | bis(dicyclohexylphosphino)ferrocene; |
| 494227-37-1 | (S,S)-(−)-2,2′-Bis[(R)-(N,N-dimethylamino)(phenyl)methyl]-1,1′- |
| | di[bis(3,5-dimethyl-4-methoxyphenyl)phosphino]ferrocene; |
| 210842-74-3 | (S,S)-(−)-2,2′-Bis[(R)-(N,N-dimethylamino)(phenyl)methyl]-1,1′- |
| | bis(diphenylphosphino)ferrocene; |
| 494227-36-0 | (S,S)-(−)-2,2′-Bis[(R)-(N,N-dimethylamino)(phenyl)methyl]-1,1′- |
| | di[bis(3,5-trifluoromethylphenyl)phosphino]ferrocene; |
| 360048-63-1 | (R)-(−)-1-[(S)-2-(Bis(3,5-dimethyl-4- |
| | methoxyphenyl)phosphino)ferrocenyl]ethyldicyclohexylphosphine; |
| | (R)-(+)-1,1′-Bis(diphenylphosphino)-2,2′-bis(N,N-di-i- |
| | propylamido)ferrocene (R)-CTH-JAFAPhos; |
| 97239-80-0 | 1,1′-Bis(di-i-propylphosphino)ferrocene; |
| 246231-79-8 | (R)-(−)-1-[(S)-2-(Bis(4- |
| | trifluoromethylphenyl)phosphino)ferrocenyl]ethyl-di-t-butylphosphine; |
| 292638-88-1 | (R)-(−)-1-[(S)-2-(Di(3,5-bis- |
| | trifluoromethlphenyl)phosphino)ferrocenyl]ethyldicyclohexylphosphine |
| 166172-63-0 | (R)-(−)-1-[(S)-2-(Di(3,5-bis- |
| | trifluoromethylphenyl)phosphino)ferrocenyl]ethyldi(3,5- |
| | dimethylphenyl)phosphine; |
| 494227-38-2 | (S)-(+)-[(R)-2-Dicyclohexylphosphinoferrocenyl](N,N- |
| | dimethylamino)(2-dicyclohexylphosphinophenyl)methane; |
| 158923-11-6 | (R)-(−)-1-[(S)-2-(Dicyclohexylphosphino)ferrocenyl]ethyldi-t- |
| | butylphosphine; |
| 158923-07-0 | (S)-(+)-1-[(R)-2- |
| | (Dicyclohexylphosphino)ferrocenyl]ethyldicyclohexylphosphine; |
| 158923-09-2 | (R)-(−)-1-[(S)-2- |
| | (Dicyclohexylphosphino)ferrocenyl]ethyldiphenylphosphine; |
| 162291-01-2 | (S)-(+)-1-[(R)-2- |
| | (Dicyclohexylphosphino)ferrocenyl]ethyldiphenylphosphine; |
| 494227-32-6 | (R)-(+)-1-[(R)-2-(2′- |
| | Dicyclohexylphosphinophenyl)ferrocenyl]ethyldi(bis-3,5- |
| | trifluoromethylphenyl)phosphine; |
| 494227-30-4 | (R)-(+)-1-[(R)-2-(2′-Di(3,5-dimethyl-4- |
| | methoxyphenyl)phosphinophenyl)ferrocenyl]ethyldi(bis-3,5- |
| | trifluoromethylphenyl)pho; |
| | (S)-(+)-1-[(R)-2-(Diphenylphosphino)ferrocenyl]ethyldi-t- |
| | butylphosphine; |
| 360048-66-4 | (R)-(+)-1-[(R)-2- |
| | (Diphenylphosphino)ferrocenyl]ethyldicyclohexylphosphine; (R)-(R)- |
| | JOSIPHOS |
| 162291-02-3 | (S)-(+)-1-[(R)-2- |
| | (Diphenylphosphino)ferrocenyl]ethyldicyclohexylphosphine ethanol |
| | adduct; (S)-(R)-JOSIPHOS |
| 387868-06-6 | (R)-(−)-1-[(R)-2-(2′-Diphenylphosphinophenyl)ferrocenyl]ethyldi(bis-3,5- |
| | trifluoromethylphenyl)phosphine; |
| 388079-60-5 | (R)-(−)-1-[(R)-2-(2′- |
| | Diphenylphosphinophenyl)ferrocenyl]ethyldicyclohexylphosphine; |
| 494227-31-5 | (R)-(+)-1-[(R)-2-(2′-Diphenylphosphinophenyl)ferrocenyl]ethyldi(3,5- |
| | xylyl)phosphine; |
| 494227-33-7 | (R)-(+)-1-[(R)-2-(2′-Di(3,5- |
| | xylyl)phosphinophenyl)ferrocenyl]ethyldi(3,5-xylyl)phosphine; |
| 312959-24-3 | 1,2,3,4,5-Pentaphenyl-1′-(di-t-butylphosphino)ferrocene; CTC-Q-PHOS |
| 15629-92-2 | 1,3-Bis(diphenylphosphino)propane nickel (II) chloride; |
| 14126-37-5 | Bis(triphenylphosphine)nickel (II) bromide; |
| 14264-16-5 | Bis(triphenylphosphine)nickel (II) chloride; |
| 13007-90-4 | Bis(triphenylphosphine)nickel dicarbonyl; |
| 67292-34-6 | Dichloro[1,1′-bis(diphenylphosphino)ferrocene]nickel (II); |
| 13859-65-9 | Tetrakis(trifluorophosphine)nickel (0); |
| 31277-98-2 | Bis[1,2-bis(diphenylphosphino)ethane]palladium (0); |
| 53199-31-8 | Bis(tri-t-butylphosphine)palladium (0); |
| 33309-88-5 | Bis(tricyclohexylphosphine)palladium (0); |
| 359803-53-5 | Chloro(di-2-norbornylphosphino)(2′-dimethylamino-1,1′-biphenyl-2- |
| | yl)palladium (II); |
| 614753-51-4 | Chloro(di-2-norbornylphosphino)(2-dimethylaminomethylferrocen-1- |
| | yl)palladium (II); |
| 172418-32-5 | trans-Di(mu-acetato)bis[o-(di-o-tolylphosphino)benzyl]dipalladium (II); |
| 14588-08-0 | Diacetatobis(triphenylphosphine)palladium (II); |
| 185812-86-6 | Di--bromobis(tri-t-butylphosphino)dipalladium (I); |
| 19978-61-1 | Dichloro(1,2-bis(diphenylphosphino)ethane)palladium (II); |
| 72287-26-4 | Dichloro[1,1′-bis(diphenylphosphino)ferrocene]palladium (II) |
| | dichloromethane adduct; |
| 29934-17-6 | trans-Dichlorobis(tricyclohexylphosphine)palladium (II); |
| 40691-33-6 | trans-Dichlorobis(tri-o-tolylphosphine)palladium (II); |
| | Buchwald Biaryl Phosphine Ligand Kit for Aromatic Carbon-Heteroatom |
| | Bond Formation and Suzuki Coupling; |
| | Phosphine Ligand Kit for Palladium-Catalyzed Carbon-Carbon and |
| | Carbon-Heteroatom Bond Formation; |
| | Acetato(2′-di-t-butylphosphino-1,1′-biphenyl-2-yl)palladium (II); |
| 395116-70-8 | Benzyldi-1-adamantylphosphine; [cataCXium ® ABn]; |
| 7650-91-1 | Benzyldiphenylphosphine; |
| 121954-50-5 | alpha,alpha′-Bis(di-t-butylphosphino)-o-xylene; |
| 37170-64-2 | 1,2-Bis(dichlorophosphino)-1,2-dimethylhydrazine; |
| 28240-68-8 | Bis(dichlorophosphino)methane; |
| 23743-26-2 | 1,2-Bis(dicyclohexylphosphino)ethane; |
| 137349-65-6 | Bis(dicyclohexylphosphino)methane; |
| 6411-21-8 | 1,2-Bis(diethylphosphino)ethane; |
| 23936-60-9 | 1,2-Bis(dimethylphosphino)ethane; |
| 64065-08-3 | Bis(dimethylphosphino)methane; |
| 76858-94-1 | 1,2-Bis(dipentafluorophenylphosphino)ethane; |
| 5112-95-8 | Bis(diphenylphosphino)acetylene; |
| 2960-37-4 | N,N-Bis(diphenylphosphino)amine; |
| 229177-79-1 | (S)-(−)-2,2′-Bis(N-diphenylphosphinoamino)-5,5′,6,6′,7,7′,8,8′-octahydro- |
| | 1,1′-binaphthyl; CTH-(S)-BINAM; |
| 13991-08-7 | 1,2-Bis(diphenylphosphino)benzene; |
| 84783-64-2 | 2,2′-Bis(diphenylphosphino)-1,1′-biphenyl; BIPHEP; |
| 398127-98-5 | R-6,6′-Bis(diphenylphosphino)-1,1′-biphenyl-2,2′-diylbis(acetate); (R)- |
| | Methyl Soniphos; |
| | S-6,6′-Bis(diphenylphosphino)-1,1′-biphenyl-2,2′-diylbis(acetate); (S)- |
| | Methyl Soniphos; |
| 398128-03-5 | R-6,6′-Bis(diphenylphosphino)-1,1′-biphenyl-2,2′- |
| | diylbis(cyclohexylcarboxylate); (R)-Cyclohexyl Soniphos; |
| | S-6,6′-Bis(diphenylphosphino)-1,1′-biphenyl-2,2′- |
| | diylbis(cyclohexylcarboxylate); (S)-Cyclohexyl Soniphos; |
| 301847-89-2 | R-(−)-1,13-Bis(diphenylphosphino)-7,8-dihydro-6H- |
| | dibenzo[f,h][1,5]dioxonin; (R)-C3-TUNEPHOS; |
| 984-43-0 | 1,2-Bis(diphenylphosphino)ethane monooxide; |
| 983-80-2 | cis-1,2-Bis(diphenylphosphino)ethylene; |
| 23582-02-7 | Bis(2-diphenylphosphinoethyl)phenylphosphine; TRIPHOS; |
| 2071-20-7 | Bis(diphenylphosphino)methane; |
| 23176-18-3 | Bis(diphenylphosphino)methane monooxide; |
| | (R)-(−)-4,12-Bis(diphenylphosphino)-[2.2]-paracyclophane; (R)- |
| | PHANEPHOS; |
| 261733-18-0 | 4,6-Bis(diphenylphosphino)phenoxazine; NIXANTPHOS; |
| 166330-10-5 | Bis(2-diphenylphosphinophenyl)ether; DPEphos; |
| 85685-99-0 | 1,3-Bis(diphenylphosphino)propane monooxide; |
| 503538-69-0 | R-(−)-5,5′-Bis(diphenylphosphino)-2,2,2′,2′-tetrafluoro-4,4′-bi-1,3- |
| | benzodioxole, dichloromethane adduct; (R)-DIFLUORPHOS TM; |
| 503538-70-3 | S-(+)-5,5′-Bis(diphenylphosphino)-2,2,2′,2′-tetrafluoro-4,4′-bi-1,3- |
| | benzodioxole, dichloromethane adduct; (S)-DIFLUORPHOS TM; |
| | R-(+)-6,6′-Bis(diphenylphosphino)-2,2′,3,3′-tetrahydro-5,5′-bi-1,4- |
| | benzodioxin; (R)-SYNPHOS TM; |
| | S-(−)-6,6′-Bis(diphenylphosphino)-2,2′,3,3′-tetrahydro-5,5′-bi-1,4- |
| | benzodioxin; (S)-SYNPHOS TM; |
| 80499-19-0 | 1,4-Bis(di-i-propylphosphino)butane; |
| | 1,2-Bis(di-4-sulfonatophenylphosphino)benzene tetrasodium salt DMSO |
| | adduct |
| | (R)-(−)-4,12-Bis(di(3,5-xylyl)phosphino)-[2.2]-paracyclophane; CTH- |
| | (R)-3,5-xylyl-PHANEPHOS; |
| | (S)-(+)-4,12-Bis(di(3,5-xylyl)phosphino)-[2.2]-paracyclophane; CTH- |
| | (S)-3,5-xylyl-PHANEPHOS; |
| 36802-41-2 | Bis(2-methoxyphenyl)phenylphosphine; |
| 28240-66-6 | 1,3-Bis(phenylphosphino)propane; |
| 151888-20-9 | Bis(p-sulfonatophenyl)phenylphosphine dihydrate dipotassium salt; |
| 65013-26-5 | Bis(triphenylphosphine)iminium borohydride; |
| 21050-13-5 | Bis(triphenylphosphine)iminium chloride; |
| 321921-71-5 | Butyldi-1-adamantylphosphine; [cataCXium A]; |
| 93634-87-8 | t-Butyldicyclohexylphosphine; |
| 6372-42-5 | Cyclohexyldiphenylphosphine; |
| 822-68-4 | Cyclohexylphosphine; |
| 131211-27-3 | Di-1-adamantylphosphine; |
| 126683-99-6 | Di-1-adamantylphosphinic chloride; |
| 138517-61-0 | (1R,2R)-(+)-1,2-Diaminocyclohexane-N,N′-bis(2′- |
| | diphenylphosphinobenzoyl); TROST LIGAND; |
| 174810-09-4 | (1R,2R)-(+)-1,2-Diaminocyclohexane-N,N′-bis(2-diphenylphosphino-1- |
| | naphthoyl); TROST LIGAND (NAPHTHYL); |
| 205495-66-5 | (1S,2S)-(−)-1,2-Diaminocyclohexane-N,N′-bis(2-diphenylphosphino-1- |
| | naplthoyl); TROST LIGAND (NAPHTHYL); |
| 13716-10-4 | Di-t-butylchlorophosphine; |
| | Di-t-butylcyclohexylphosphine; |
| 6002-40-0 | Di-t-butylmethylphosphine; |
| 4006-38-6 | Di-i-butylphosphine; |
| 255836-67-0 | racemic-2-Di-t-butylphosphino-1,1′-binaphthyl; |
| 224311-51-7 | 2-(Di-t-butylphosphino)biphenyl; |
| 224311-49-3 | 2-Di-t-butylphosphino-2′-(N,N-dimethylamino)biphenyl; |
| 360577-23-7 | [2-(Di-t-butylphosphino)ethyl]trimethylammonium chloride; |
| 255837-19-5 | 2-Di-t-butylphosphino-2′-methylbiphenyl; |
| 1809-19-4 | Di-n-butylphosphite; |
| 185913-97-7 | (R)-(+)-5,5′-Dichloro-6,6′-dimethoxy-2,2′-bis(diphenylphosphino)-1,1′- |
| | biphenyl; (R)-Cl-MeO-BIPHEP; |
| 185913-98-8 | (S)-(−)-5,5′-Dichloro-6,6′-dimethoxy-2,2′-bis(diphenylphosphino)-1,1′- |
| | biphenyl; (S)-Cl-MeO-BIPHEP; |
| 247940-06-3 | 2-(Dicyclohexylphosphino)biphenyl; |
| | 2-Dicyclohexylphosphino-2′,6′-dimethoxy-1,1′-biphenyl; S-PHOS; |
| 213697-53-1 | 2-(Dicyclohexylphosphino)-2′-(N,N-dimethylamino)biphenyl; |
| 181864-78-8 | [2-(Dicyclohexylphosphino)ethyl]trimethylammonium chloride; |
| 251320-86-2 | 2-Dicyclohexylphosphino-2′-methylbiphenyl; |
| 564483-18-7 | 2-(Dicyclohexylphosphino)-2′,4′,6′-tri-i-Propyl-1,1′-biphenyl; X-PHOS; |
| 39864-68-1 | Dicyclopentylphosphine; |
| 627-49-6 | Diethylphosphine; |
| 762-04-9 | Diethylphosphite; |
| 21302-09-0 | Dilauryl hydrogen phosphite; |
| 161265-03-8 | 9,9-Dimethyl-4,5-bis(diphenylphosphino)xanthene; XANTPHOS; |
| 672-66-2 | Dimethylphenylphosphine; |
| 148432-44-4 | Di-2-norbornylphosphine; |
| 1499-21-4 | Diphenylphosphinic chloride; |
| 186803-02-1 | (1R,1′R,2R,2′R)-(−)-2,2′-Diphenylphosphino-1,1′-bicyclopentyl; (R,R)- |
| | BICP; |
| | (1S,1′S,2S,2′S)-(+)-2,2′-Diphenylphosphino-1,1′-bicyclopentyl; (S,S)- |
| | BICP; |
| 240417-00-9 | 2-Diphenylphosphino-2′-(N,N-dimethylamino)biphenyl; |
| 145964-33-6 | (R)-(+)-2-(Diphenylphosphino)-2′-methoxy-1,1′-binaphthyl; (R)-MOP; |
| 134484-36-9 | (S)-(−)-2-(Diphenylphosphino)-2′-methoxy-1,1′-binaphthyl; (S)-MOP; |
| 132682-77-0 | 2-Diphenylphosphino-6-methylpyridine; |
| 149341-34-4 | (R)-(+)-1-(2-Diphenylphosphino-1-naphthyl)isoquinoline (R)-QUINAP |
| 149341-33-3 | (S)-(−)-1-(2-Diphenylphosphino-1-naphthyl)isoquinoline (S)-QUINAP; |
| 164858-78-0 | (R)-(+)-2-[2-(Diphenylphosphino)phenyl]-4-(1-methylethyl)-4,5- |
| | dihydrooxazole; |
| 1809-20-7 | Di-i-propylphosphite; |
| 1017-60-3 | Di-p-tolylphosphine; |
| 607-01-2 | Ethyldiphenylphosphine; |
| 15715-41-0 | Methyldiethoxyphosphine; |
| 1486-28-8 | Methyldiphenylphosphine; |
| 43077-29-8 | (S)-(+)-Neomenthyldiphenylphosphine (S)-NMDPP; |
| 824-72-6 | Phenyldichlorophosphine oxide; |
| | N-Phenyl-2-(di-t-butylphosphino)pyrrole; [cataCXium PtB]; |
| | N-Phenyl-2-(dicyclohexylphosphino)pyrrole; [cataCXium PCy]; |
| 2946-61-4 | Phenyldimethoxyphosphine; |
| 1779-48-2 | Phenylphosphinic acid; |
| 15573-31-6 | n-Propyldichlorophosphine; |
| 362524-23-0 | (S)-(−)-2,2′,6,6′-Tetramethoxy-4,4′-bis(diphenylphosphino)-3,3′- |
| | bipyridine; CTH-(S)-P-PHOS; |
| 442905-33-1 | (R)-(+)-2,2′,6,6′-Tetramethoxy-4,4′-bis(di(3,5-xylyl)phosphino)-3,3′- |
| | bipyridine; CTH-(R)-Xylyl-P-Phos; |
| 3676-97-9 | Tetramethylbiphosphine disulfide; |
| 1031-93-2 | p-Tolyldiphenylphosphine; |
| 16523-89-0 | Triallylphosphine; |
| 4125-25-1 | Tri-i-butylphosphine; |
| 13716-12-6 | Tri-t-butylphosphine; |
| 102-85-2 | Tri-n-butylphosphite; |
| 29949-85-7 | Tri(m-chlorophenyl)phosphine; |
| 1159-54-2 | Tri(p-chlorophenyl)phosphine; |
| 7650-88-6 | Tricyclopentylphosphine; |
| 554-70-1 | Triethylphosphine; |
| 122-52-1 | Triethylphosphite; |
| 594-09-2 | Trimethylphosphine; |
| 82164-75-8 | 2,4,4-Trimethylpentylphosphine; |
| 594-09-2 | Trimethylphosphine; |
| 3411-48-1 | Tri(1-naphthyl)phosphine; |
| 14540-52-4 | Tri-neo-pentylphosphite; |
| 4731-53-7 | Tri-n-octylphosphine; TOP; |
| 101-02-0 | Triphenylphosphite; |
| 6476-36-4 | Tri-i-propylphosphine, |
| 2234-97-1 | Tri-n-propylphosphine; |
| 116-17-6 | Tri-i-propylphosphite; |
| 51805-45-9 | Tris(2-carboxyethyl)phosphine, hydrochloride; |
| 4023-53-4 | Tris(2-cyanoethyl)phosphine; |
| 31570-04-4 | Tris(2,4-di-t-butylphenyl)phosphite; |
| 443150-11-6 | Tris(4,6-dimethyl-3-sulfanatophenyl)phosphine trisodium salt hydrate; |
| 28926-65-0 | 1,1,1-Tris(diphenylphosphino)methane; |
| 22031-12-5 | 1,1,1-Tris(diphenylphosphinomethyl)ethane; |
| 2767-80-8 | Tris(hydroxymethyl)phosphine; |
| 4706-17-6 | Tris(hydroxypropyl)phosphine; |
| 121898-64-4 | Tris(4-methoxy-3,5-dimethylphenyl)phosphine; |
| 4731-65-1 | Tris(o-methoxyphenyl)phosphine; |
| 29949-84-6 | Tris(m-methoxyphenyl)phosphine; |
| 1259-35-4 | Tris(pentafluorophenyl)phosphine; |
| 63995-70-0 | Tris(3-sulfonatophenyl)phosphine hydrate; |
| 13406-29-6 | Tris(p-trifluoromethylphenyl)phosphine; |
| 91608-15-0 | Tris (2,4,6-trimethoxyphenyl)phosphine; |
| 15573-38-3 | Tris(trimethylsilyl)phosphine; |
| 6224-63-1 | Tri-m-tolylphosphine; |
| 2155-96-6 | Vinyldiphenylphosphine; |
| 60648-70-6 | Bis(tri-t-butylphosphine)platinum (O); |
| 15692-07-6 | cis-Dichlorobis(triethylphosphine)platinum (II); |
| 15604-36-1 | cis-Dichlorobis(triphenylphosphine)platinum (II); |
| 19529-53-4 | Tetrakis(trifluorophosphine)platinum (0) (ampouled under PF3); |
| 23032-93-1 | Iododioxobis(triphenylphosphine)rhenium (V); |
| 154661-57-1 | (2S,4S)-(−)-2,4-Bis(diphenylphosphino)pentane(norbornadiene)rhodium |
| | (I) hexafluorophosphate; |
| 157772-65-1 | 1,1′-Bis(di-i-propylphosphino)ferrocene(1,5-cyclooctadiene)rhodium (I) |
| | tetrafluoroborate; |
| 13938-94-8 | Chlorocarbonylbis(triphenylphosphine)rhodium (I); |
| 17185-29-4 | Hydridocarbonyltris(triphenylphosphine)rhodium (I); |
| 25360-32-1 | Carbonyl(dihydrido)tris(triphenylphosphine)ruthenium (II); |
| | Chloro[(R)-(+)-5,5′-dichloro-6,6′-dimethoxy-2,2′- |
| | bis(diphenylphosphino)-1,1′-biphenyl](p-cymene)ruthenium (II) chloride |
| | CH2C12 adduct; |
| | Chloro[(S)-(−)-5,5′-dichloro-6,6′-dimethoxy-2,2′-bis(diphenylphosphino)- |
| | 1,1′-biphenyl](p-cymene)ruthenium (II) chloride CH2C12 adduct; |
| 141686-21-7 | Chloro[hydrotris(pyrazol-1-yl)borato]bis(triphenylphosphine)ruthenium |
| | (II) ethanol adduct; |
| 99897-61-7 | Chloro(indenyl)bis(triphenylphosphine)ruthenium (II), dichloromethane |
| | adduct; |
| 329735-86-6 | Dichloro[(R)-(+)-2,2′-bis(diphenylphosphino)-1,1′-binaphthyl][(2R)-(−)- |
| | 1,1-bis(4-methoxyphenyl)-3-methyl-1,2-butanediamine]ruthenium (II); |
| 329735-87-7 | Dichloro[(S)-(−)-2,2′-bis(diphenylphosphino)-1,1′-binaphthyl][(1R,2R)- |
| | (+)-1,2-diphenylethylenediamine]ruthenium (II); |
| 329736-05-2 | Dichloro[(S)-(−)-2,2′-bis(diphenylphosphino)-1,1′-binaphthyl][(1S,2S)-(−)- |
| | 1,2-diphenylethylenediamine]ruthenium (II); |
| 325150-57-0 | Dichloro[(R)-(−)-4,12-bis(di(3,5-xylyl)phosphino)-[2.2]- |
| | paracyclophane][(1S,2S)-(−)-1,2-diphenylethylenediamine]ruthenium (II); |
| 14564-35-3 | Dichlorodicarbonylbis(triphenylphosphine)ruthenium (II); |
| 148630-66-4 | Trimethylphosphine(hexafluoroacetylacetonato)silver (I); |
| 82149-18-6 | Sodium(cyclopentadienyl)tris(dimethylphosphito)cobaltate (I); |
| 10039-56-2 | Sodium hypophosphite monohydrate; |
| | [[(2S,3S,5S,6S)-5,6-dimethoxy-5,6-dimethyl-1,4-dioxane-2,3- |
| | diyl]bis(methylene)]bis[diphenyl-Phosphine |
| | [[(2R,3R,5R,6R)-5,6-dimethoxy-5,6-dimethyl-1,4-dioxane-2,3- |
| | diyl]bis(methylene)]bis[diphenyl-Phosphine |
| | 1,1″-[(1R,2R)-1,2-cyclohexanediylbis(iminocarbonyl)]bis[2- |
| | (diphenylphosphino)-, (2R,2″R)-Ferrocene |
| | 1,1″-[(1S,2S)-1,2-cyclohexanediylbis(iminocarbonyl)]bis[2- |
| | (diphenylphosphino)-, (2S,2″S)-Ferrocene |
| | 2,5-bis[bis[3,5-bis(trifluoromethyl)phenyl]phosphino]-1,2,5,6- |
| | tetradeoxy-3,4-O-(1-methylethylidene)-D-Mannitol |
| | 2,5-bis[bis[3,5-dimethylphenyl)phosphino]-1,2,5,6-tetradeoxy-3,4-O-(1- |
| | methylethylidene)-D-Mannitol |
| | Phosphinous acid, diphenyl-, (1S)-3,3′-diphenyl[1,1′-binaphthalene]-2,2′- |
| | diyl ester |
| | Phosphinous acid, diphenyl-, (1R)-3,3′-diphenyl[1,1′-binaphthalene]-2,2′- |
| | diyl ester |
| | 1,1′-bis(1,1-dimethylethyl)-, (1S,1′S,2R,2′R)-2,2′-Biphospholane |
| | 1,1′-bis(1,1-dimethylethyl)-, (1R,1′R,2S,2′S)-2,2′-Biphospholane |
| | [(2,3,5,6-η)-bicyclo[2.2.1]hepta-2,5-diene][(1R,1′R,2R,2′R)-1,1′-bis(1,1- |
| | dimethylethyl)-2,2′-biphospholane-κP1,κP1′]-, (OC-6-11)-Rhodium(1+) |
| | hexafluoroantimonate(1−) |
| | [(2,3,5,6-η)-bicyclo[2.2.1]hepta-2,5-diene][(1R,1′R,2R,2′R)-1,1′-bis(1,1- |
| | dimethylethyl)-2,2′-biphospholane-κP1,κP1′]-Rhodium(1+) |
| | [1,1′-bis[(3aS,4S,6S,6aS)-tetrahydro-2,2,4,6-tetramethyl-5H- |
| | phospholo[3,4-d]-1,3-dioxol-5-yl-κP5]ferrocene][(1,2,5,6-η)-1,5- |
| | cyclooctadiene]-, hexafluorophosphate(1−) Rhodium(1+) |
| | [1,1′-bis[(3aS,4S,6S,6aS)-tetrahydro-2,2,4,6-tetramethyl-5H- |
| | phospholo[3,4-d]-1,3-dioxol-5-yl-κP5]ferrocene][(1,2,5,6-η)-1,5- |
| | cyclooctadiene]-Rhodium(1+) |
| | 1,1′-bis[(3aS,4S,6S,6aS)-tetrahydro-2,2,4,6-tetramethyl-5H- |
| | phospholo[3,4-d]-1,3-dioxol-5-yl]-Ferrocene |
| | 1,1′-bis[(3aS,4S,6S,6aS)-tetrahydro-2,2-dimethyl-4,6-bis(phenylmethyl)- |
| | 5H-phospholo[3,4-d]-1,3-dioxol-5-yl]-Ferrocene |
| | 1,1′-bis[(3aS,4S,6S,6aS)-4,6-diethyltetrahydro-2,2-dimethyl-5H- |
| | phospholo[3,4-d]-1,3-dioxol-5-yl]-Ferrocene, |
| | 1,1′-bis[(11bS)-3,5-dihydro-4H-dinaphtho[2,1-c:1′,2′-e]phosphepin-4-yl]- |
| | Ferrocene |
| | 1,1′-bis[(11bR)-3,5-dihydro-4H-dinaphtho[2,1-c:1′,2′-e]phosphepin-4-yl]- |
| | Ferrocene, |
| | 3,3′-Bi-3H-dinaphtho[2,1-c:1′,2′-e]phosphepin, 4,4′-bis(1,1- |
| | dimethylethyl)-4,4′,5,5′-tetrahydro-, (3S,3′S,4S,4′S,11bS,11′bS)- |
| | [(1″S)-4′,4″,5′,5″,6′,6″-hexamethoxy[1,1′:3′,1″:3″,1′′′-quaterphenyl]-2′,2″- |
| | diyl]bis[diphenyl-Phosphine |
| | 5,5′-(1,2-phenylene)bis[tetrahydro-2,2,4,6-tetramethyl-, |
| | (3aS,3′aS,4R,4′R,6R,6′R,6aS,6′aS)-5H-Phospholo[3,4-d]-1,3-dioxole |
| | 5,5′-(1,2-phenylene)bis[tetrahydro-2,2,4,6-tetramethyl-, |
| | (3aS,3′aS,4S,4′S,6S,6′S,6aS,6′aS)-5H-Phospholo[3,4-d]-1,3-dioxole |
| | 5,5′-(1,2-phenylene)bis[tetrahydro-2,2-dimethyl-4,6-bis(phenylmethyl)- |
| | 5H-Phospholo[3,4-d]-1,3-dioxole |
| | 5,5′-(1,2-phenylene)bis[tetrahydro-2,2-dimethyl-4,6-bis(3-methylbutyl)- |
| | 5H-Phospholo[3,4-d]-1,3-dioxole |
| | 5,5′-(1,2-phenylene)bis[tetrahydro-2,2-dimethyl-4,6-dipropyl-5H- |
| | Phospholo[3,4-d]-1,3-dioxole |
| | 5,5′-(1,2-phenylene)bis[4,6-diethyltetrahydro-2,2-dimethyl-, |
| | (3aS,3′aS,4S,4′S,6S,6′S,6aS,6′aS)-5H-Phospholo[3,4-d]-1,3-dioxole |
| | 4,4′-(1,2-phenylene)bis[4,5-dihydro-, (11bS,11′bS)-3H-Dinaphtho[2,1- |
| | c:1′,2′-e]phosphepin |
| | 4,4′-(1,2-phenylene)bis[4,5-dihydro-, (11bR,11′bR)-3H-Dinaphtho[2,1- |
| | c:1′,2′-e]phosphepin |
| | Ruthenate(1−), tri-μ-chlorodichlorobis[(11aS)-dibenzo[d,f][1,3]dioxepin- |
| | 1,11-diylbis[diphenylphosphine-κP]]di-, hydrogen, compd. with N- |
| | methylmethanamine (1:1) |
| | Ruthenate(1−), tri-μ-chlorodichlorobis[(11aS)-dibenzo[d,f][1,3]dioxepin- |
| | 1,11-diylbis[diphenylphosphine-κP]]di-, hydrogen |
| | (11aS)-dibenzo[d,f][1,3]dioxepin-1,11-diylbis[diphenyl-Phosphine |
| | (11aR)-dibenzo[d,f][1,3]dioxepin-1,11-diylbis[diphenyl-Phosphine |
| | Ruthenate(1−), tri-μ-chlorodichlorobis[[(12aS)-6,7- |
| | dihydrodibenzo[e,g][1,4]dioxocin-1,12-diyl]bis[diphenylphosphine- |
| | κP]]di-, hydrogen, compd. with N-methylmethanamine (1:1) |
| | Ruthenate(1−), tri-μ-chlorodichlorobis[[(12aS)-6,7- |
| | dihydrodibenzo[e,g][1,4]dioxocin-1,12-diyl]bis[diphenylphosphine- |
| | κP]]di-, hydrogen, |
| | [(12aS)-6,7-dihydrodibenzo[e,g][1,4]dioxocin-1,12-diyl]bis[diphenyl- |
| | Phosphine |
| | [(12aR)-6,7-dihydrodibenzo[e,g][1,4]dioxocin-1,12-diyl]bis[diphenyl- |
| | Phosphine |
| | [(13aS)-7,8-dihydro-6H-dibenzo[f,h][1,5]dioxonin-1,13- |
| | diyl]bis[diphenyl-Phosphine |
| | [(13aR)-7,8-dihydro-6H-dibenzo[f,h][1,5]dioxonin-1,13- |
| | diyl]bis[diphenyl-Phosphine |
| | [(14aS)-6,7,8,9-tetrahydrodibenzo[b,d][1,6]dioxecin-1,14- |
| | diyl]bis[diphenyl-Phosphine |
| | [(14aR)-6,7,8,9-tetrahydrodibenzo[b,d][1,6]dioxecin-1,14- |
| | diyl]bis[diphenyl-Phosphine |
| | [(15aR)-7,8,9,10-tetrahydro-6H-dibenzo[b,d][1,6]dioxacycloundecin- |
| | 1,15-diyl]bis[diphenyl-Phosphine |
| | [(15aS)-7,8,9,10-tetrahydro-6H-dibenzo[b,d][1,6]dioxacycloundecin- |
| | 1,15-diyl]bis[diphenyl-Phosphine |
| | [(16aR)-6,7,8,9,10,11-hexahydrodibenzo[b,d][1,6]dioxacyclododecin- |
| | 1,16-diyl]bis[diphenyl-Phosphine |
| | [(16aS)-6,7,8,9,10,11-hexahydrodibenzo[b,d][1,6]dioxacyclododecin- |
| | 1,16-diyl]bis[diphenyl-Phosphine |

In another embodiment, the present invention provides a composition comprising a compound selected from Table 1 and paraffin.

1. A stable composition for water and oxygen sensitive compounds comprising: (a) at least one water and/or oxygen sensitive compound; and (b) at least one inert material with a melting point ranging from 20° C. to 250° C.

2. The stable composition of claim 1, wherein said inert material is paraffin.

3. A stable composition comprising (a) a compound selected from Table 1; and (b) at least one inert material with a melting point ranging from 20° C. to 250° C.

4. The stable composition of claim 3, wherein said inert material is paraffin.

5. A stable composition comprising (a) 1,3-(bis(mesityl)-2-imidazolidinylidene)-dichloro-(phenylmethylene)(tricyclohexylphosphine)ruthenium; and (b) parffin.
